(19) United States
(12) Patent Application Publication
(10) Pub. No.: US 2007/0129549 A1
(43) Pub. Date: Jun. 7, 2007

(54) Stable lamotrigine pharmaceutical compositions and processes for their preparation

(76) Inventors: Yatendra Kumar, Haryana (IN); Mohan Prasad, Haryana (IN); Asok Nath, Haryana (IN); Nitin Maheshwari, Delhi (IN)

Correspondence Address: RANBAXY INC., 600 COLLEGE ROAD EAST, SUITE 2100, PRINCETON, NJ, 08540, US

(21) Appl. No.: 10/550,173

(22) PCT Filed: Mar. 22, 2004
(86) PCT No.: PCT/IB04/00843

§ 371 Date: Dec. 4, 2006

(30) Foreign Application Priority Data
Mar. 21, 2003 (IN) 352/DEL/2003

(51) Int. Cl. C07D 211/06

(52) U.S. Cl. 546/206

(57) ABSTRACT

The invention relates to processes for the preparation of piperidylmethyl-indanones, and to the use of these compounds as intermediates for the preparation of benzyl-piperidylmethyl-indanones which are active compounds for the treatment of CNS disorders. The invention also relates to a process for the preparation of donepezil or a pharmaceutically acceptable salt thereof, and pharmaceutical compositions that include the donepezil or a pharmaceutically acceptable salt thereof.

### FIELD OF THE INVENTION

The field of the invention relates to processes for the preparation of piperidylmethyl-indanones, and to the use of these compounds as intermediates for the preparation of benzyl-piperidylmethyl-indanones which are active compounds for the treatment of CNS disorders. The invention also relates to a process for the preparation of donepezil or a pharmaceutically acceptable salt thereof, and pharmaceutical compositions that include the donepezil or a pharmaceutically acceptable salt thereof.

### BACKGROUND OF THE INVENTION

Benzyl-piperidylmethyl-indanones such as donepezil have an excellent pharmacological action as prophylactic or medicament for senile dementia, especially for Alzheimer disease. Several processes have been reported for the preparation of benzyl-piperidylmethyl-indanones for example, in U.S. Pat. Nos. 4,895,841; 5,606,064; 6,252,081; 6,413,986; WO 97/22584 and J. Med. Chem. 1995, 38(24), 4821-4829. These processes require multiple steps or complicated purification processes such as chromatography and therefore inevitably lead to poorer yields or purity.

### SUMMARY OF THE INVENTION

In one general aspect there is provided a process for preparing 2-(4-piperidinyl)methyl-1-indanone of formula II, or a salt thereof,

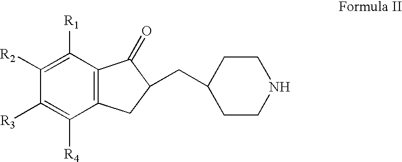

wherein R¹, R², R³, and R⁴ are identical or different, and represent hydrogen or straight or branched-chain alkyl, alkoxy, alkoxycarbonyl, alkyl- or dialkyl-aminocarbonyloxy, trifluoromethyl, or halogen. The process includes reducing 2-(4-pyridyl)methyl-1-indanone of the formula III, or a salt thereof,

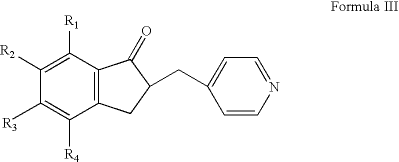

wherein R¹, R², R³, and R⁴ are as defined above; and recovering the 2-(4-piperidinyl)methyl-1-indanone of formula II, or a salt thereof.

Recovering the 2-(4-piperidinyl)methyl-1-indanone may include one or more of distillation, distillation under vacuum, filtration, filtration under vacuum, decantation and centrifugation.

The process may include further drying of the product obtained.

In another general aspect there is provided a process for preparing the 2-(4-pyridyl)methyl-1-indanone of formula III, or a salt thereof, wherein R¹, R², R³, and R⁴ are as defined above. The process includes selectively reducing 2-(4-pyridyl)methylene-1-indanone of formula IV, or a salt thereof,

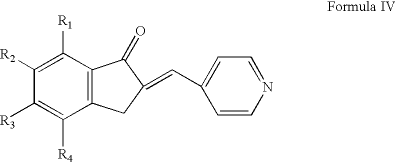

wherein R¹, R², R³, and R⁴ are as defined above; and recovering the 2-(4-pyridyl)methyl-1-indanone of formula III, or a salt thereof.

Recovering the 2-(4-pyridyl)methyl-1-indanone may include one or more of distillation, distillation under vacuum, filtration, filtration under vacuum, decantation and centrifugation.

The process may include further drying of the product obtained.

In another general aspect there is provided a process for preparing benzyl-piperidylmethyl-indanones of formula I, or a salt thereof,

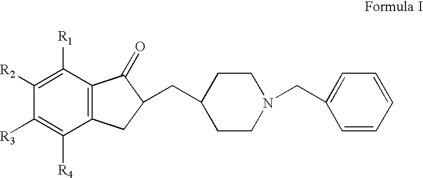

wherein R¹, R², R³, and R⁴ are as defined above.

The process includes reacting the 2-(4-piperidinyl)methyl-1-indanone of formula II, or a salt thereof, with a benzyl derivative of formula V,

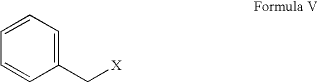

wherein X is a leaving group, in the presence of a base; and recovering the benzyl-piperidylmethyl-indanones, or a salt thereof.

Recovering the benzyl-piperidylmethyl-indanones may include one or more of distillation, distillation under vacuum, filtration, filtration under vacuum, decantation and centrifugation.

The process may include further drying of the product obtained.

The Compounds of formula I may thus be obtained in good yield and purity without resorting to chromatographic purification.

In another general aspect there is provided an improved process for preparing donepezil of formula VI, or a pharmaceutically acceptable salt thereof.

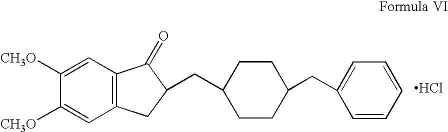

The process includes reacting 2-(4-piperidinyl)methyl-1-indanone of formula II, or a salt thereof, wherein R¹ and R⁴ represent hydrogen and R² and R³ represent methoxy, with a benzyl derivative of formula V, wherein X is a leaving group, in the presence of an inorganic base and a phase transfer catalyst.

In another general aspect there is provided a pharmaceutical composition that includes a therapeutically effective amount of donepezil or a pharmaceutically acceptable salt thereof, and one or more pharmaceutically acceptable carriers, excipients or diluents.

The details of one or more embodiments of the inventions are set forth in the description below. Other features, objects and advantages of the inventions will be apparent from the description and claims.

### DETAILED DESCRIPTION OF THE INVENTION

The inventors have developed an efficient process for the preparation of 2-(4-piperidinyl)methyl-1-indanone of formula II, or a salt thereof, wherein R¹, R², R³, and R⁴ are identical or different, and represent hydrogen, or straight or branched-chain alkyl, alkoxy, alkoxycarbonyl, alkyl- or dialkyl-aminocarbonyloxy, trifluoromethyl, or halogen. The process involves reducing 2-(4-pyridyl)methyl-1-indanone of formula III, or a salt thereof.

Examples of alkyl groups include methyl, ethyl, n-propyl, isopropyl, and tert-butyl. Examples of alkoxy groups include methoxy, ethoxy, isopropoxy, and tert-butoxy. The term “halogen” includes fluorine, chlorine, bromine, and iodine. Examples of alkoxylcarbonyl include methoxycarbonyl, ethoxycarbonyl, isopropoxycarbonyl, and tert-butoxycarbonyl. Examples of alkyl- or dialkyl-aminocarbonyloxy include methylaminocarbonyloxy, and dimethylaminocarbonyloxy. In a particular example, R¹ and R⁴ represent hydrogen and R² and R³ represent methoxy in the compounds of formula II and III.

In general, the reduction may be achieved by hydrogenation in the presence of a catalyst. The hydrogenation catalysts used for the reduction are the customary hydrogenation catalysts known in organic chemistry, for example transistion metal compounds. Examples of transistion metal compounds include platinum compounds such as platinum oxide, ruthenium compounds such as ruthenium oxide and rhodium compounds such as rhodium/carbon.

The hydrogenation may be carried out at normal pressure, or at elevated pressure depending on the choice of catalyst. In general, it may be carried out at a hydrogen pressure in the range from 1 to 10 atmospheres, or at a hydrogen pressure in the range from 1 to 2 atmospheres.

The hydrogenation may be carried out at a temperature from about −20° C. to about 120° C., for example from about 0° C. to about 80° C. In particular, it may be carried out at a temperature from about 10° C. to about 35° C.

The compounds of formula II can be produced by methods known in the art such as the procedures disclosed in U.S. Pat. No. 6,413,986; WO 97/22584, J. Med. Chem. 1995, 38 (24), 4821-4829, or obtained by the reduction of compounds of formula III.

The inventors have also developed a process for the preparation of 2-(4-pyridyl)methyl-1-indanone of formula III, or a salt thereof, wherein R¹, R², R³, and R⁴ are identical or different, and represent hydrogen, or straight or branched-chain alkyl, alkoxy, alkoxycarbonyl, alkyl- or dialkyl-aminocarbonyloxy, trifluoromethyl, or halogen. The process involves selectively reducing 2-(4-pyridyl)methylene-1-indanone of formula IV, or a salt thereof.

Examples of alkyl groups include methyl, ethyl, n-propyl, isopropyl, and tert-butyl. Examples of alkoxy groups include methoxy, ethoxy, isopropoxy, and tert-butoxy. The term “halogen” includes fluorine, chlorine, bromine, and iodine. Examples of alkoxylcarbonyl include methoxycarbonyl, ethoxycarbonyl, isopropoxycarbonyl, and tert-butoxycarbonyl. Examples of alkyl- or dialkyl-aminocarbonyloxy include methylaminocarbonyloxy, and dimethylaminocarbonyloxy. In a particular example, R¹ and R⁴represent hydrogen and R² and R³ represent methoxy in the compounds of formula III and IV.

The compounds of formula III can be produced by methods known in the art such as the procedures disclosed in U.S. Pat. No. 6,252,081 or may be obtained by the selective reduction of compounds of formula IV.

In general, the reduction of the compound of formula IV to compound of formula III may be achieved by selective hydrogenation in the presence of a catalyst or by other conventional procedures for carbon-carbon double bond reduction, which do not reduce the pyridine ring of the compound of formula IV.

The inventors have observed that the formation of a complex mixture of impurities is minimized or eliminated altogether by avoiding direct reduction of the compound of formula IV to the compound of formula II.

In general, the reduction may be achieved by hydrogenation in the presence of a catalyst. The catalysts used for the selective hydrogenation are the customary hydrogenation catalysts known in organic chemistry, for example transistion metal compounds, used under milder conditions. Examples of suitable transistion metal compounds include platinum compounds such as platinum/carbon, palladium compounds such as palladium/carbon, palladium hydroxide, and nickel compounds such as Raney nickel.

The selective hydrogenation may be carried out at normal pressure, or at somewhat elevated pressure depending on the choice of catalyst. In general, it may be carried out at a hydrogen pressure in the range from 1 to 5 atmospheres, or at a hydrogen pressure in the range from 1 to 2 atmospheres.

The hydrogenation temperature may be varied depending on the choice of catalyst and/or pressure employed. For example, the hydrogenation may be carried out at a temperature from about−20° C. to about 60° C., or at a temperature from about 0° C. to about 40° C. In particular, it may be carried out at a temperature from about 10° C. to about 35° C.

The conventional procedures for selective carbon-carbon double bond reduction, which may be employed, include using hydrazine hydrate or ammonium formate/formic acid.

The compounds of formula IV are known compounds, and can be produced by methods known in the art such as the procedure disclosed in U.S. Pat. No. 5,606,064, example 1.

Suitable solvents for hydrogenation of the compounds of formula II or IV are the customary inert solvents that do not change under the reaction conditions. Examples of such solvents include ethers, such as dibutyl ether, methyl tert-butyl ether, dioxane and tetrahydrofuran; alcohols such as methanol, ethanol, propanol, isopropanol and butanol; chlorinated hydrocarbons such as dichloromethane, tetrachloromethane and dichloroethylene; esters such as ethyl acetate and isopropyl acetate; ketones such as acetone and MIBK (methylisobutylketone); hydrocarbons such as hexane, toluene, and xylene; water; polar aprotic solvents such as dimethylformamide; dimethyl sulphoxide; N-methylpyrrolidone; and mixtures thereof.

The 2-(4-piperidinyl)methyl-1-indanone of formula II, or a salt thereof so obtained may be benzylated with a benzyl derivative of formula V, in the presence of an inorganic base and a phase transfer catalyst to give benzyl-piperidylmethyl-indanones of formula I, or a salt thereof.

The inventors have observed that the benzylation rection is faster in the presence of a phase transfer catalyst and side products are minimized. The compound of formula I may thus be obtained in good yield and purity without resorting to chromatographic purification.

The phase transfer catalysts used for preparing benzyl-piperidylmethyl-indanones of formula I, are not limited, including, for example, quaternary ammonium salts, and quaternary phosphonium salts. Examples of quaternary ammonium salts include tetramethylammonium iodide, tetrabutylammonium iodide, benzyltributylammonium bromide, 1-methylpiridinium iodide, teramethyl-2-butylammonium chloride, trimethylcyclopropylammonium chloride, tetrabutylammonium bromide, and t-butylethyldimethylammonium bromide. Examples of quaternary phosphonium salts include tributylmethylphosphonium iodide, triethylmethylphosphonium iodide, methyltriphenoxyphosphonium iodide, tetrabutylphosphonium bromide, benzyltriphenylphosphonium bromide, and tetraphenylphosphonium chloride.

The benzylation reaction may be carried out in a suitable solvent.

The term “suitable solvent” includes any solvent or solvent mixture which are inert and do not change the reaction. Examples of such solvents include water; ethers such as diethyl ether, dibutyl ether, methyl tert-butyl ether, dioxane and tetrahydrofuran; chlorinated hydrocarbons such as dichloromethane, and dichloroethylene; esters such as ethyl acetate and isopropyl acetate; ketones such as acetone and MIBK (methylisobutylketone); alcohols such as methanol, ethanol, propanol and isopropanol; acetonitrile; dimethylformamide; dimethyl sulphoxide; 1,2-dimethoxyethane; N-methylpyrrolidone; sulpholane; and mixtures thereof.

The temperature at which the benzylation reaction may be carried out may range from about −20° C. to about 120° C., for example from about 0° C. to about 40° C. In particular, it may be carried out at a temperature from about 10° C. to about 35° C.

The base used for preparing a benzyl-piperidylmethyl-indanones of formula I, includes, for example an amine, an inorganic base or ammonia. Examples of amines include triethylamine, N-methyl morpholine, N,N-dimethyl benzyl amine, pyridine, picoline, and lutidine.

The inorganic base may be an alkali metal carbonate, bicarbonate or hydroxide. Examples of alkali metal carbonates include lithium carbonate, potassium carbonate and sodium carbonate. Examples of alkali metal bicarbonates include potassium bicarbonate and sodium bicarbonate. Examples of alkali metal hydroxides include potassium hydroxide and sodium hydroxide.

The product obtained may be further or additionally dried to achieve the desired moisture values. For example, the product may be further or additionally dried in a tray drier, dried under vacuum and/or in a Fluid Bed Drier.

The present invention is further illustrated by the following examples which are provided merely to be exemplary of the invention and are not intended to limit the scope of the invention.

Preparation 1

### Preparation of 5,6-dimethoxy-2-(pyridine-4-yl)methylene-indan-1-one

A mixture of 5,6-dimethoxy-indan-1-one (10 g), pyridine-4-carboxaldehyde (67 g), p-toluene sulfonic acid (118 g) in toluene (1200 ml) was refluxed azeotropically for 6 hours. The reaction mixture was cooled to room temperature and filtered. The wet solid so obtained was stirred with 10% aqueous sodium carbonate solution. The solid was filtered, washed with acetone and then dried to get the title compound (130 g).

HPLC Purity: 99.5%

### EXAMPLE 1

### Preparation of 5,6-dimethoxy-2-(4-pyridyl)methyl-indan-1-one

5,6-dimethoxy-2-(pyridine-4-yl) methylene-indan-1-one (10 g, from preparation 1) was hydrogenated using 10% Palladium/carbon (10 g, 50% moisture) in a mixture of methanol (1500 ml) and methylene chloride (1000 ml) at atomospheric pressure. The hydrogen gas was bubbled into the reaction mixture for about 5 hours. The reaction mixture was filtered and the filtrate was concentrated to get the title compound (92 g).

HPLC Purity: 99.8%.

### EXAMPLE 2

### Prepration of 2,3-dihydro-5,6-dimethoxy-2-(4-piperidinyl)methyl-indan-1-one, hydrochloride

A mixture of 5,6-dimethoxy-2-(4-pyridyl)methyl-indan-1-one (25 g from example 1), methanol (125 ml), water (125 ml), conc. hydrochloric acid(12.5 g) and platinum dioxide (2.5 g) was hydrogenated at 15 to 20 psi hydrogen pressure for about 6 hours. The reaction mixture was filtered, the filtrate was concentrated and the residue so obtained was crystallized from methanol to get the title compound (24 g).

HPLC Purity: 99.4%.

### EXAMPLE 3

### Preparation of 1-benzyl-4-((5,6-dimethoxy-1-indanone)-2-yl)methylpiperidine, hydrochloride (donepezil hydrochloride)

To a stirred mixture of 2,3-dihydro-5,6-dimethoxy-2-(4-piperidinyl)methyl-indan-1-one, hydrochloride (10 g from example 2), tetrabutyl ammonium bromide (1 g), potassium carbonate (9 g) in a mixture of water (40 ml) and methylene chloride (50 ml) was added benzyl bromide (5.3 g) at 20-25° C. over 30 minutes. After the addition was over, the reaction mixture was stirred at the same temperature for about 30 minutes. The organic layer was separated and stirred with a mixture of water (20 ml) and conc. hydrochloric acid (6.4 g) at 20-25° C. for 15 minutes. The organic layer was separated and concentrated. The residue so obtained was dissolved in water (100 ml) and extracted with ethyl acetate (50 ml). The organic layer was discarded and pH of the aqueous layer was adjusted to 9.5 with aqueous ammonia solution (3.5 ml). The aqueous solution was then extracted with ethyl acetate (50 ml). The ethyl acetate extract was washed with water. The organic layer was concentrated and the residue was dissolved in methanol (50 ml). To this solution, conc. hydrochloric acid (4.8 g) diluted with methanol (10 ml) was added. Diisopropylether (120 ml) was then added to the solution at 25° C. It was further stirred at 5 to 10° C. and the separated solid was filtered and dried to get crystals of donepezil hydrochloride (10 g).

HPLC Purity: 99.95%.

While the present invention has been described in terms of its specific embodiments, certain modifications and equivalents will be apparent to those skilled in the art and are intended to be included within the scope of the present invention.

1. A process for the preparation of 2-(4-piperidinyl)methyl-1-indanone of formula II, or a salt thereof, 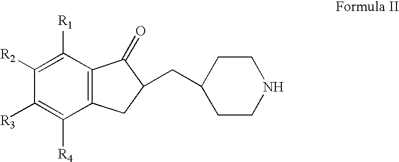
wherein R¹, R², R³, and R⁴ are identical or different, and represent hydrogen, straight or branched-chain alkyl, alkoxy, alkoxycarbonyl, alkyl- or dialkyl-aminocarbonyloxy, trifluoromethyl, or halogen,
the process comprising reducing 2-(4-pyridyl)methyl-1-indanone of formula III, or a salt thereof, 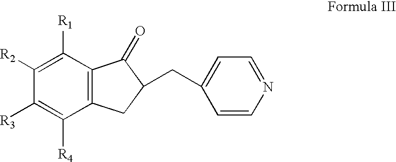
wherein R¹, R², R³, and R⁴ are as defined above; and recovering the 2-(4-piperidinyl)methyl-1-indanone of formula II.

2. The process of claim 1, wherein R¹ and R⁴ represent hydrogen and R² and R³ represent methoxy in formula II and formula III.

3. The process of claim 1, wherein the reduction comprises hydrogenation in the presence of a catalyst.

4. The process of claim 3, wherein the catalyst comprises one or more of platinum oxide, ruthenium oxide, and rhodium/carbon.

5. The process of claim 3, wherein the hydrogenation is carried out at a pressure of from about 1 to about 2 atmospheres using hydrogen gas.

6. The process of claim 3, wherein the hydrogenation is carried out at a temperature of from about 10° C. to about 35° C.

7. The process of claim 3, wherein the hydrogenation is carried out in a solvent.

8. The process of claim 7, wherein the solvent comprises one or more of ethers, alcohols, chlorinated hydrocarbons, esters, ketones, hydrocarbons, polar aprotic solvents, water and mixtures thereof.

9.-15. (canceled)

16. The process of claim 1, wherein the recovering comprises one or more of distillation, distillation under vacuum, filtration, filtration under vacuum, decantation, and centrifugation.

17. A process for the preparation of 2-(4-pyridyl)methyl-1-indanone of formula III, or a salt thereof, 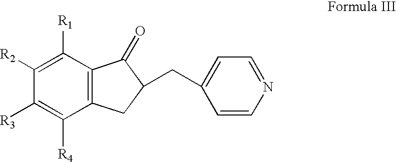
wherein R¹, R², R³, and R⁴ are identical or different, and represent hydrogen, straight or branched-chain alkyl, alkoxy, alkoxycarbonyl, alkyl- or dialkyl-aminocarbonyloxy, trifluoromethyl, or halogen, the process comprising selectively reducing 2-(4-pyridyl)methylene-1-indanone of formula IV, or a salt thereof, 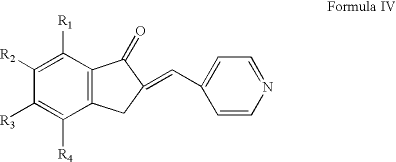
wherein R¹, R², R³, and R⁴ are as defined above; and recovering the 2-(4-pyridyl)methyl-1-indanone of formula III.

18. The process of claim 17, wherein R¹ and R⁴ represent hydrogen and R² and R³ represent methoxy in formula III and formula IV.

19. The process of claim 17, wherein the reduction comprises hydrogenation in the presence of a catalyst.

20. The process of claim 17, wherein the catalyst comprises one or more of palladium/carbon, platinum/carbon and Raney nickel.

21. The process of claim 17, wherein the hydrogenation is carried out at a temperature of from about 10° C. to about 35° C.

22. The process of claim 17, wherein the hydrogenation is carried out in a solvent.

23. The process of claim 22, wherein the solvent comprises one or more of ethers, alcohols, chlorinated hydrocarbons, esters, ketones, hydrocarbons, polar aprotic solvents, water, and mixtures thereof.

24.-30. (canceled)

31. The process of claim 17, wherein the recovering comprises one or more of distillation, distillation under vacuum, filtration, filtration under vacuum, decantation, and centrifugation.

32. A process for the preparation of benzyl-piperidylmethyl-indanones of formula I, or a salt thereof, 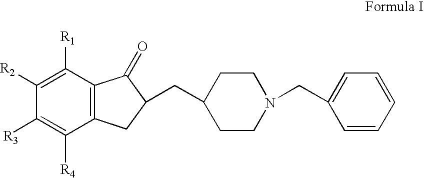
wherein R¹, R², R³, and R⁴ are identical or different, and represent hydrogen, straight or branched-chain alkyl, alkoxy, alkoxycarbonyl, alkyl- or dialkyl-aminocarbonyloxy, trifluoromethyl, or halogen,
the process comprising reacting 2-(4-piperidinyl)methyl-1-indanone of the formula II, or a salt thereof, prepared by the process of claim 1, with a benzyl derivative of formula V, 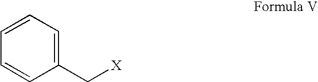
wherein X is a leaving group; and recovering the benzyl-piperidylmethyl-indanones of formula I.

33. The process of claim 32, wherein the leaving group X in the benzyl derivative of formula V is chloride, bromide, iodide, tosylate, or sulphate.

34. The process of claim 32, wherein the reaction is carried out in the presence of a base and a phase transfer catalyst.

35. The process of claim 34, wherein the base comprises one or more of an amine, an inorganic base and ammonia.

36. The process of claim 35, wherein the inorganic base is an alkali metal carbonate.

37. The process of claim 36, wherein the alkali metal carbonate comprises one or more of lithium carbonate, potassium carbonate and sodium carbonate.

38. The process of claim 34, wherein the phase transfer catalyst is comprises one or more of quaternary ammonium salt, or quaternary phosphonium salt.

39. The process of claim 38, wherein the quaternary ammonium salt comprises one or more of tetramethylammonium iodide, tetrabutylammonium iodide, teramethyl-2-butylammonium chloride, trimethylcyclopropylammonium chloride, tetrabutylammonium bromide, and t-butylethyldimethylammonium bromide.

40. The process of claim 32, wherein the reaction is carried out at a temperature of from about 0° C. to about 40° C.

41. The process of claim 32, wherein the reaction is carried out in a solvent.

42. The process of claim 41, wherein the solvent comprises one or more of ethers, alcohols, chlorinated hydrocarbons, esters, ketones, hydrocarbons, polar aprotic solvents, water and mixtures thereof.

43.-49. (canceled)

50. The process of claim 32, wherein the recovering comprises one or more of distillation, distillation under vacuum, filtration, filtration under vacuum, decantation, and centrifugation.

51. A process for the preparation of donepezil of formula VI or a pharmaceutically acceptable salt thereof, 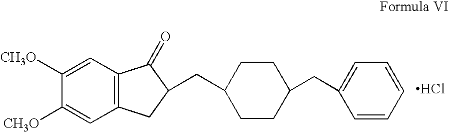
the process comprising:
(a) selectively reducing 2-(4-pyridyl)methylene-1-indanone of formula IV, or a salt thereof, 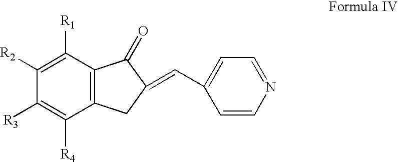
to obtain 2-(4-pyridyl)methyl-1-indanone of formula III, 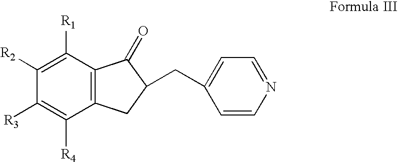
wherein R¹ and R⁴ represent hydrogen and R² and R³ represent methoxy in formula III and formula IV,
(b) reducing the 2-(4-pyridyl)methyl-1-indanone of formula III to obtain 2-(4-piperidinyl)methyl-1-indanone of formula II, 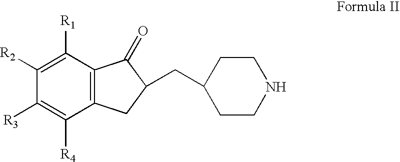
wherein R¹ and R⁴ represent hydrogen and R² and R³ represent methoxy,
(c) reacting the 2-(4-piperidinyl) methyl-1-indanone of formula II, with a benzyl derivative of formula V, 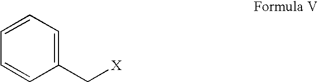
wherein X is a leaving group, in the presence of an inorganic base and a phase transfer catalyst, and
(d) recovering the donepezil or a pharmaceutically acceptable salt thereof.

52. The process of claim 51, wherein the leaving group X in the benzyl derivative of formula V is chloride, bromide, iodide, tosylate, or sulphate.

53. A pharmaceutical composition comprising a therapeutically effective amount of donepezil or a pharmaceutically acceptable salt thereof obtained by the process of claim 51; and one or more pharmaceutically acceptable carriers, excipients or diluents.
